(19) United States
(12) Patent Application Publication
(10) Pub. No.: US 2003/0216691 A1
(43) Pub. Date: Nov. 20, 2003

(54) Laproscopic pump

(75) Inventor: Kirk Jacobson, Marco Island, FL (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Endo-AID, Inc.

(21) Appl. No.: 10/150,347

(22) Filed: May 17, 2002

(51) Int. Cl. A61M 37/00

(52) U.S. Cl. 604/148

(57) ABSTRACT

An apparatus has two principal components: a pressure driver, typically a gas filled cartridge, canister, container or the like, that is activated by opening of a membrane, typically a metal foil by puncturing or rupturing, and a holder, that maintains the pressure driver in an air tight chamber. When the cartridge is opened, the pressurized gas exits the cartridge and enters the air tight chamber, causing it to expand. This expansion creates pressure on the portion of the apparatus holding a fluid filled container, and ultimately forces fluid out from the container, typically into tubing, so that the fluid reaches a patient. A simple valving device, typically in a handpiece, and typically proximate the surgical site, can be used at the end of the tubing to regulate the fluid flow into the patient. Systems employing this apparatus are also disclosed, as are method of use for the aforementioned apparatus and systems.

### TECHNICAL FIELD

The systems, apparatus and methods disclosed herein are directed to pumps and in particular disposable pumps for pumping irrigant or drug solutions into a patient.

### BACKGROUND

Laproscopic surgery is a very common form of surgery. This is because it is minimally invasive and patients typically have shorter hospital stays and recovery times, when compared with conventional open surgery. One aspect of laproscopic surgery involves introducing irrigation fluid to the surgical site, from a fluid source.

This fluid source is typically a bag, filled with the requisite irrigating fluid. The bag is connected to a pump, with the bag typically at a distance of approximately one to three meters from the surgical site and outside of the surgical field. The pump connects to a tube, which delivers irrigation fluid to the surgical site. The bag and pump are normally elevated above patient level, to utilize gravity in combination with pumping for delivery of the irrigation fluid.

This system exhibits drawbacks. Besides being gravity dependent, as the bag must be hung above patient level, the pump must be primed, either by gravity and/or, in most cases, hand pressure, prior to the surgical procedure, so that irrigation fluid reaches the surgical site upon the first demand from the surgeon. As the result of using a pump, irrigation fluid contacts the pump components and can be subject to contamination.

Additionally, these pumps have electrical cables, that can provide electrical shocks to the person attending to them. Finally, these pumps use batteries, in particular, eight AA alkaline batteries, which are exhausted after each procedure. These batteries must be disposed of in an environmentally safe manner.

### SUMMARY

The system, apparatus and methods disclosed herein overcome the deficiencies in the contemporary art, as there is provided an apparatus that can operate independent of height and altitude in delivering irrigation fluid to the surgical site. The apparatus and system disclosed herein are made of low cost components and are light weight, thus allowing for portability of the apparatus and systems.

The system provides a driver that places sufficient force on the irrigation fluid source such that irrigation fluid can be delivered to the surgical site, via tubing, without the assistance of gravity, and in many cases against gravity. The driver is external to the fluid source and tubing, and provides force on the fluid source to deliver fluid to the surgical site, through the tubing, at the requisite pressures, e.g. approximately 150 mHg to 900 mmHg.

Since the driver is external to the fluid source, as it is typically housed completely within the apparatus, it does not contact any fluid, and along with the fluid source being directly connected to the tube (tubing) in a sterile manner, the possibility of fluid contamination is negligible. Absent any pump components, such as impellers or the like, that are typically associated with the aforementioned conventional electrical pumps, any preparation or priming is minimized or eliminated altogether.

Additionally, the driver is typically a gas filled cylinder, with environmentally safe gases, such as carbon dioxide and the like under pressure, which operates manually. This eliminates any need for pumps and thus, the need for batteries. Since electrical components are not present, chances for shocking and electrical buildup are nonexistent. Moreover, as the driver is typically a gas filled cylinder housed within the apparatus, there are not any cords or cables, extending from it, as with conventional pumps and/or pump components.

The apparatus of the system has two principal components: a pressure driver, typically a gas filled cartridge, canister, container or the like, that is activated by opening of a membrane, typically a metal foil by puncturing or rupturing, and a holder, that maintains the pressure driver in an air tight chamber. When the cartridge is opened, the pressurized gas exits the cartridge and enters the air tight chamber, causing it to expand. This expansion creates pressure on the portion of the holder holding the fluid filled container, and ultimately forces fluid in the container into the tubing towards the patient. A simple valving device, typically in a handpiece, and typically proximate the surgical site, can be used at the end of the tubing to regulate the fluid flow into the patient.

Also disclosed is a fluid delivery apparatus having an activatable driver, for example, a cartridge with gas under pressure, a first section for at least partially housing the activatable driver, a second section in communication with the first section, the second section configured for holding at least one fluid container, and a partition intermediate the first section and the second section. The partition is and movable in response to forces from the driver after the driver has been activated. This partition is typically elastic.

There is also disclosed a system for fluid delivery having a fluid delivery apparatus, a valving unit for controlling fluid flow to a patient, and a first conduit in communication with the fluid delivery apparatus and the valving unit. The fluid delivery apparatus includes an activatable driver, for example, a cartridge with gas under pressure, a first section for at least partially housing the activatable driver, a second section in communication with the first section, the second section configured for holding at least one fluid container, and a partition intermediate the first section and the second section. The partition is movable in response to forces from the driver after the driver has been activated.

The system can also include a suction source, and a second conduit in communication with the valving unit and the suction source. It can also have a handpiece configured for manual manipulation, the handpiece including the valving unit.

There is disclosed a method for providing fluid to a mammalian body, for example, a human patient, that includes providing fluid delivery apparatus, the delivery apparatus having an activatable driver, a first section for at least partially housing the activatable driver, a second section in communication with the first section, the second section including at least one fluid container, and a partition intermediate the first section and the second section. The partition is movable in response to forces from the driver after the driver has been activated. A pathway for fluid in the fluid delivery apparatus to the mammalian body is created. The driver is then activated. The fluid delivery apparatus can be positioned at, above or below, the level of the mammalian body, as it is not altitude dependent and delivers fluids at pressures sufficient so as not to require the assistance of gravity.

### BRIEF DESCRIPTION OF THE DRAWINGS

Attention is now directed to the drawing figures, where like reference numerals or characters indicate corresponding or like components. In the drawings:

FIG. 1 is a perspective view of the system disclosed herein in an exemplary operation;

FIG. 2 is an exploded view of the apparatus of FIG. 1;

FIGS. 3A-3C are a cross-sectional views of the apparatus of FIGS. 1 and 2 as taken along lines 3A-3A, 3B-3B and 3C-3C, various components removed;

FIG. 4A is an exploded view of an alternate embodiment of the present invention;

FIG. 4B is a cross sectional view of the alternate embodiment of FIG. 4A taken along line 4B-4B;

FIG. 5A is a cross-sectional view of an embodiment of the apparatus including components;

FIG. 5B is a cross-sectional view of another embodiment of the apparatus including components; and

FIGS. 6 and 7 are cross-sectional views of the apparatus of FIG. 5A in operation.

### DETAILED DESCRIPTION OF THE DRAWINGS

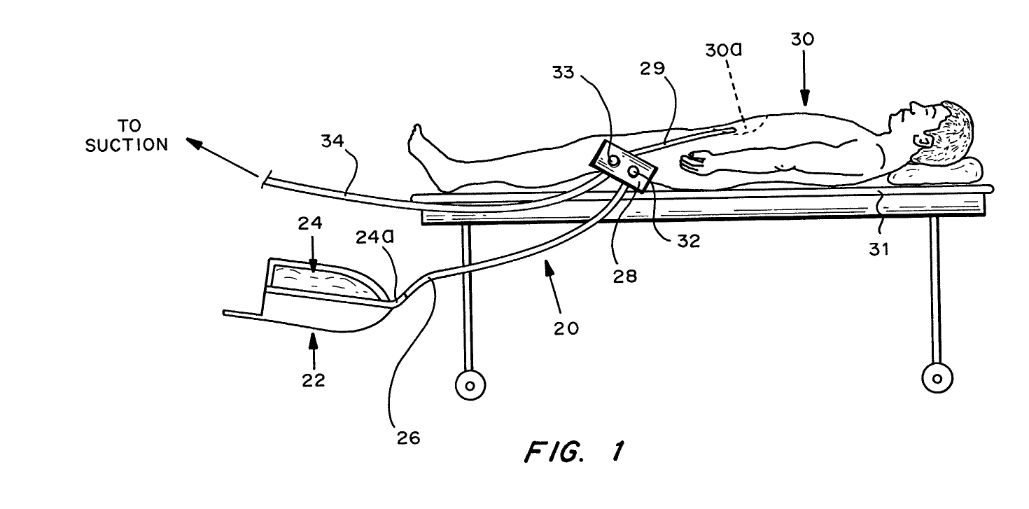

FIG. 1 shows the system 20 disclosed herein in an exemplary operation. The system 20 includes an apparatus 22 holding a fluid source 24, and delivering the fluid to the desired site, as the apparatus 22 functions as a pump. The fluid source 24, for example, is a bag (or other suitable container) containing irrigating fluid such as 0.9N saline, phosphate buffered saline, dextrose, or saline, and is connected to a tube (tubing) 26, by conventional sterile connections. The tube 26 is positioned at or near the surgical site, and is typically coupled with a valving unit 28 and ingress/egress line 29, for providing irrigation fluid to the patient 30, for example to the abdomen 30a, for example, in association with laproscopic procedures. Here, the apparatus 22 is below the level of the table 31 supporting the patient 30, as the force on the irrigation fluid to be pumped from the fluid source 24 is at pressures sufficient for properly irrigating the surgical site. The fluid source 24 can also be positioned above patient level, typically on an Intravenous (IV) pole or the like, or at the level of the patient 30,

The valving unit 28, is typically a handpiece, operated by a surgeon or other medical professional, with buttons 32, 33 for controlling fluid flow into the patient 30 as well as suction, through a suction tube 34 from a suction source (not shown).

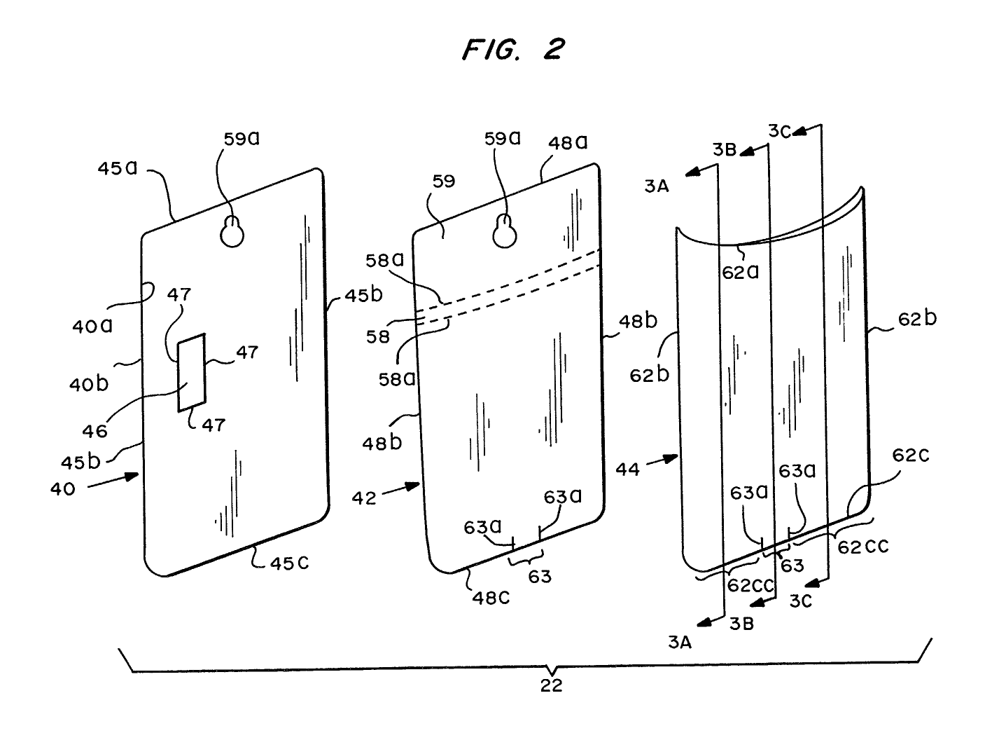

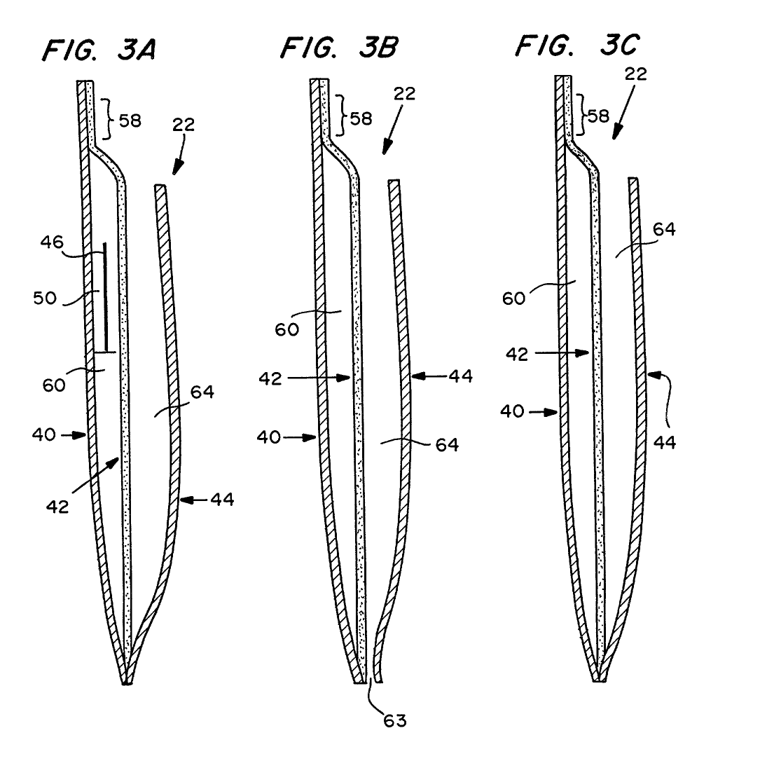

FIGS. 2 and 3A-3C detail the apparatus 22, typically formed from three layers 40, 42, 44. These layers 40, 42, 44 are described hereinafter with reference to their typical orientations for an exemplary apparatus 22. These orientations, and therefore terms such as top, bottom, upper and lower, are exemplary only, so as to assist in the description below. These layers include a base layer 40, an intermediate layer 42, over the base layer 40, and a top layer 44, over the intermediate layer 42.

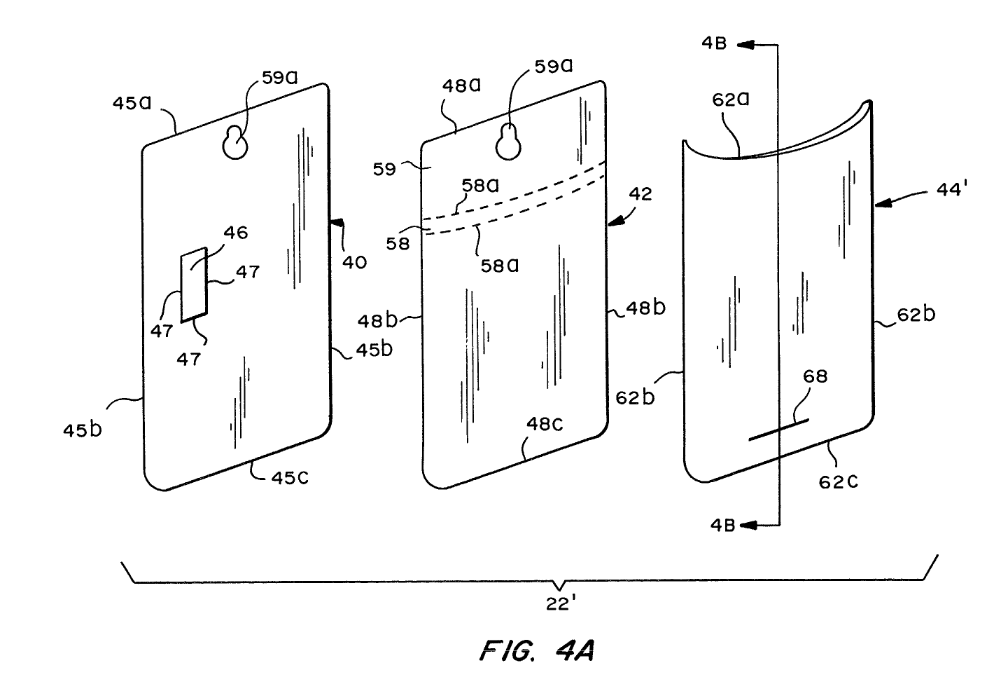

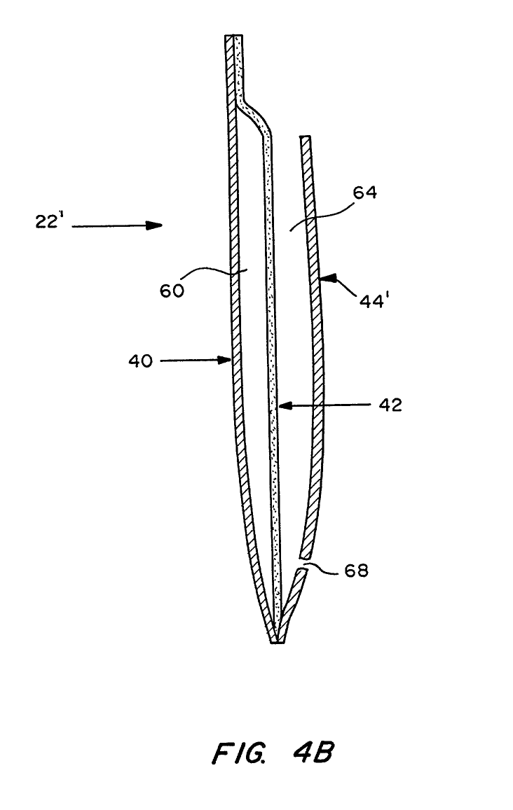

The base layer 40 has inner 40a and outer 40b sides and top 45a, lateral 45b and bottom 45c edges. A sub-layer 46 is joined to the inner 40a side of the base layer 40, typically along three sides 47. This joining, for example, by a radio frequency (RF) double weld, defines a pocket 50 (FIG. 3A), dimensioned to accommodate a gas cartridge 70 (FIG. 4) canister, container or the like, and retain the cartridge 70 therein in a frictionally tight and snug manner. The cartridge 70 can be additionally secured in the pocket 50 by adhesives, mechanical fasteners or the like.

The intermediate layer 42, is typically of the same size and dimensions as the base layer 40, and is typically joined to the base layer 40 along substantial portions thereof, and typically, the entire periphery, formed by upper 48a, lateral 48b and lower 48c edges, contacting the corresponding upper 45a, lateral 45b and lower 45c edges of the base layer 40, as well as along an intermediate area 58, typically defined as the area between broken lines 58a (drawn for emphasis only). There is an area 59 between the intermediate area 58 and the upper edge 48a, with an opening 59a extending through the base 40 and intermediate layers 42, to accommodate hanging structures, for example, those of an Intravenous (IV) pole or the like. This joining along the intermediate area 58 results in an air tight chamber 60 between the base layer 40 and the intermediate layer 42.

The top layer 44 is dimensioned to be joined to the portions of the periphery of the intermediate layer 42 (and the base layer 40). One side 62a, typically the top side, is typically open (not joined to the intermediate layer 42), while the lateral sides 62b and opposite side 62c, typically the bottom side is joined to the intermediate/base layers, except for an opening 63 between hash marks 63a (shown for emphasis only). The joined segments 62 cc along this bottom side 62b serve as stop surfaces or confinements for the fluid source 24, while the opening 63 is dimensioned for the neck portion 24a of the fluid source 24, tubes, or for providing access to the fluid source 24.

This joining of the top layer 44 to the intermediate 42/base 40 layers defines a pocket 64 between the top and layer 44 and the intermediate layer 42. The pocket 64 is dimensioned to accommodate the fluid source 24, for example a bag of fluid, such as irrigation fluid or other fluids (including those detailed below), in a frictionally tight or snug manner.

An alternate apparatus 22′, as shown in FIGS. 4A and 4B, this top layer 44′ could be joined along three sides 62b, 62c to corresponding sides/edges 48b, 48c of the intermediate layer 42. An opening 68, for example formed by a slit, would extend through this top layer 44′, and be dimensioned to accommodate the neck portion of the fluid source 24, to which the tube 26 is connected. In this alternate apparatus 22′, all other components are similar to those detailed above, and labeled accordingly.

All of the aforementioned layers 40, 42, 44, 46 are typically formed of polymeric materials such as polyvinylchloride (PVC), other plastics or elastomers, typically in sheets, that allow for minimal stretching of the material. These layers 40, 42, 44, 46 are typically joined to each other by methods including radio frequency (RF) welding, for example, the RF weld may be a double RF weld, as well as other conventional material joining techniques.

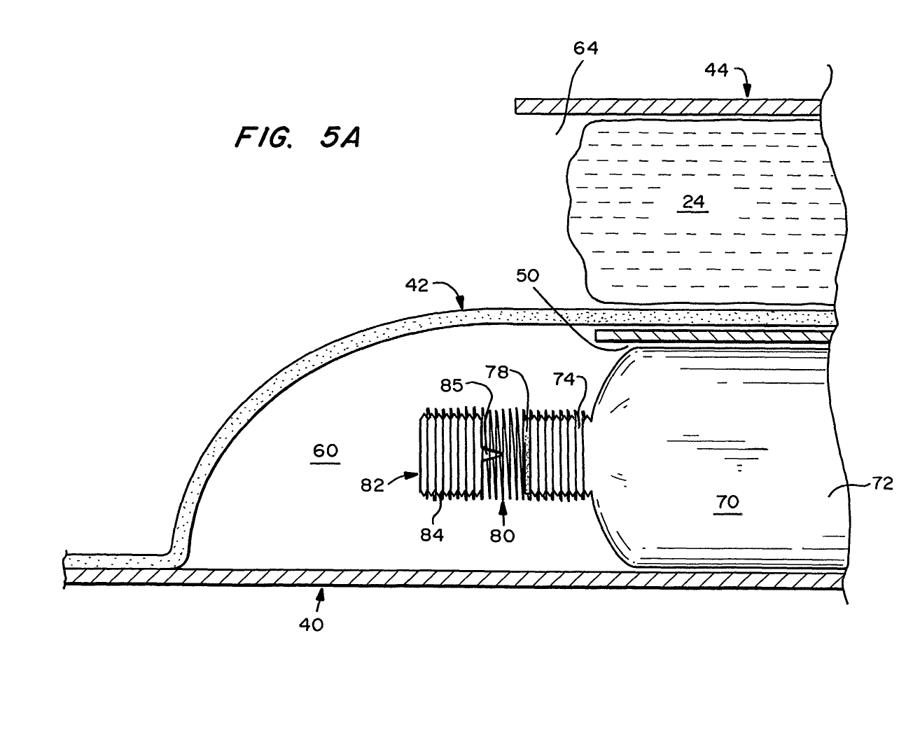

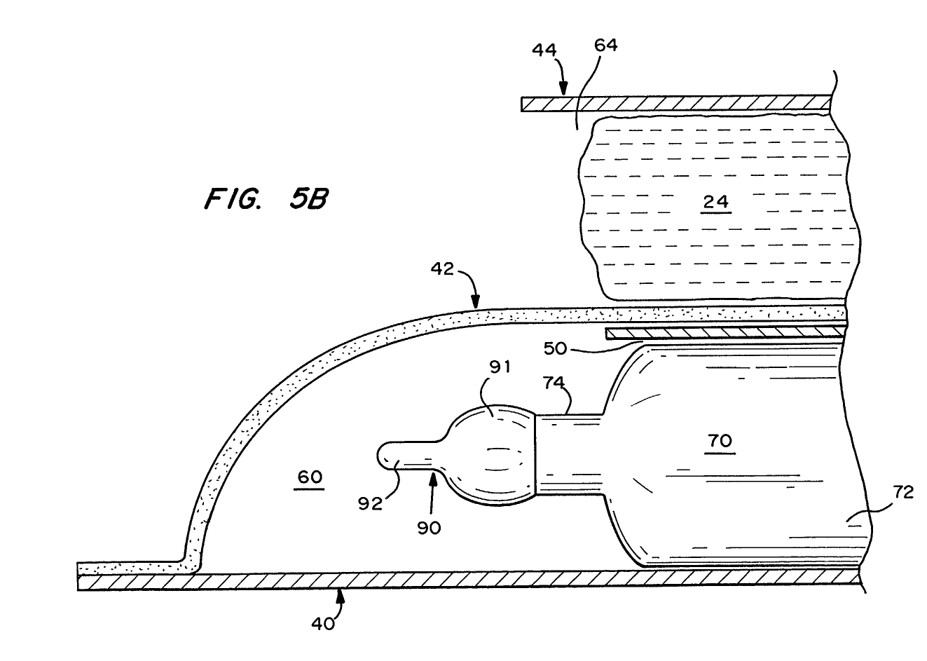

FIG. 5A shows the apparatus 22 with the fluid source 24 in the pocket 64 and the gas cartridge 70 (and associated puncturing mechanisms, as detailed below) in place in the pocket 50 in the chamber 60. The gas cartridge 70 is typically a standard canister for holding compressed gasses such as CO₂, oxygen, and air or the like, for example, in pressurized volumes of approximately 12-16 grams. For example, a suitable gas cartridge for use here is a LEYLAND™ 16 gms CO₂ filled Cartridge MIL-C-6016 Type. The cartridge 70 is formed from a body 72 and a neck 74, with the neck 74, and covered by a puncturable or rupturable seal 78. This seal 78 can be, for example, a foil or other membrane (metal or polymeric), typically welded or otherwise joined to the neck 74 of the cartridge 70, or by other standard CO₂ filled cartridge manufacturing techniques. The cartridge 70, when its seal 78 is punctured, functions as a driver for the apparatus 22 and system 20 as detailed below.

The neck 74 is typically threaded, for example, in a ⅜-24 threading, to receive a portion of a compression spring 80, in a frictionally tight manner, as the wire of the spring 80 partially seats in the threads of the neck 74. This compression spring 80 is typically made of metal, such as stainless steel, but other materials including polymers are also suitable.

The other portion of the spring 80 receives a rod 82, with a head 84 and a pointed tail 85. The rod 82 is also typically threaded (for example, also of a ⅜-24 threading), to retain the spring 80 in a frictionally tight manner, as the wire of the spring sits partially in the threads of the rod 82. The spring 80 aligns the rod 82 in with the seal 78, typically under tension to reduce the amount of pressure needed to rupture the seal 78. When activation is desired, the rod 82 can be moved toward the cartridge 70, such that the tail 85 punctures or ruptures the seal 78 (allowing gas to exit the cartridge 70, as detailed below), with the pressure of the gas escaping the cartridge 70 sufficient to move past the rod 82 and inflate the chamber 60. The rod 82, is typically made of steel, but could also be made of plastic or other suitable materials.

Alternately, as shown in FIG. 5B, the cartridge 70 could be covered by a cap 90, for example in a “bowling pin” or “gourd” shape, so as to include a body 91 and a neck 92, thinned than the body 91 and extending therefrom. This cap 90 is typically a piece of metal, such as solder or the like, placed into a sealing position on the cartridge 70 by sealing techniques such as soldering, welding, or the like. The cap 90 typically includes There may are also weakened portions cut into or indented into this cap 90, allowing it to be broken off by sufficient force from fingers, tools or the like. Alternately, the cap 90 can be made of polymers or the like.

When activation is desired, the user feels for the cap 90, and in particular the neck 92, and moves it with his thumb or other finger (or tool if desired), such that the cap 90 at least partially separates from the cartridge neck 74, allowing gas to exit the cartridge 70 (and ultimately fill the chamber 60, such that fluid is forced from the fluid source 24 to the patient 30, as detailed below).

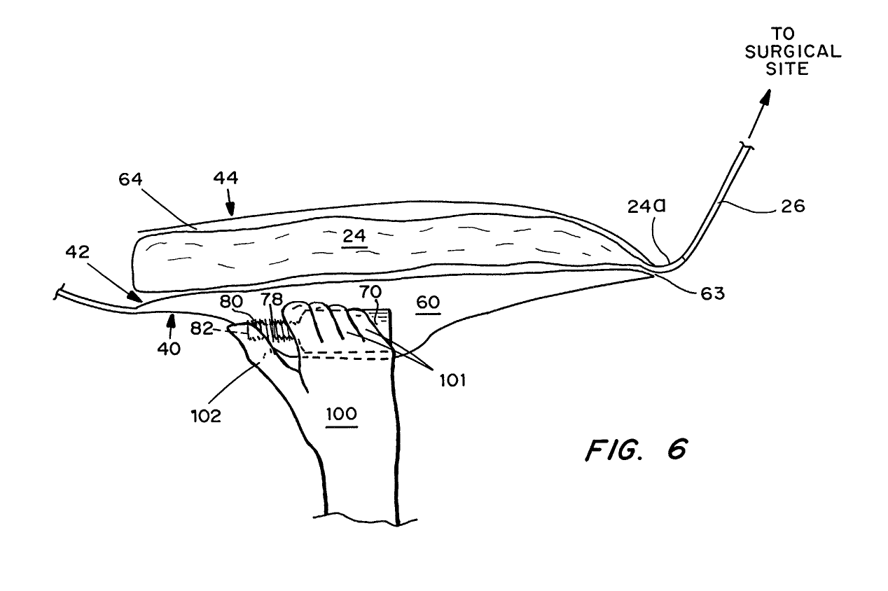

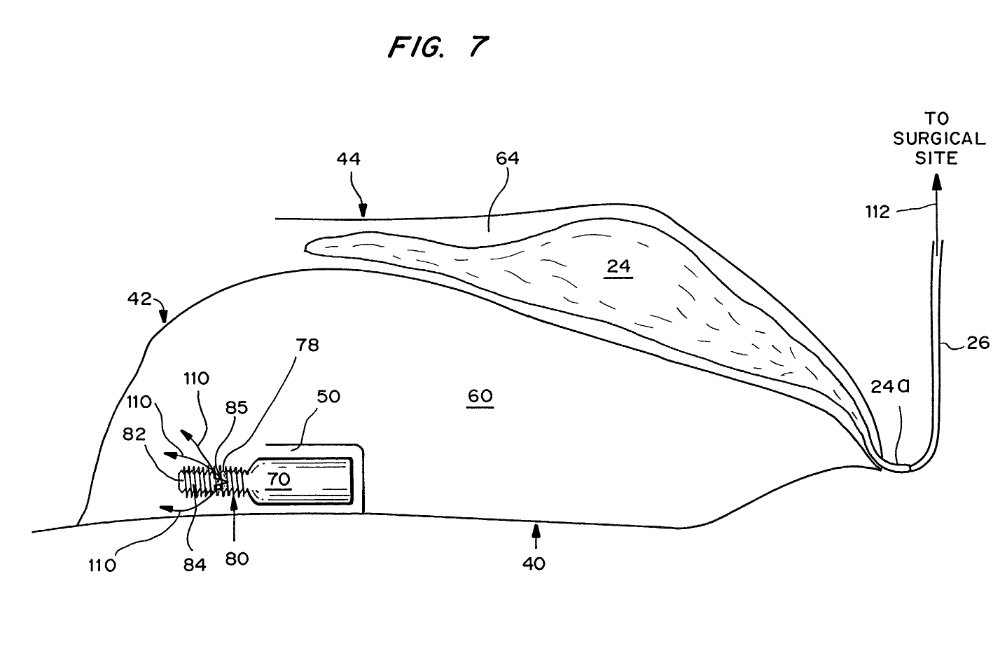

In exemplary operation, the apparatus 22, as shown in FIGS. 1, 2, 3A-3C, and 5A in conjunction with the system 20, as shown in FIG. 1, is initially inactive, in accordance with FIG. 5A as detailed above. When activation is desired, as shown in FIG. 6, the user firmly grasps their hand 100 onto the apparatus 22, and locates the pin 82, in particular its head 84. The user, typically grasps the cartridge 70 with their fingers 101, and preferably, with their thumb 102, then presses the head 84 toward the cartridge 70. This pressing of the head 84 continues as the tail 85 penetrates through the seal 78, perforating, puncturing or rupturing it, opening the cartridge 70.

The user then releases the head 84, with gas escaping the cartridge 70 at pressures great enough to escape through the puncture and around the rod 82, as illustrated by the arrows 110. The escaping gas expands into the chamber 60, filling it and moving the intermediate layer 42, functioning as a bladder, into a pressing contact with the bag of the fluid source 24. This pressure forces fluid from the fluid source 24 into the tubing 26 and ultimately to the surgical site, in the direction of the arrow 112. The cartridge 70 has a gas supply sufficient to empty an entire standard bag of irrigation fluid, for example a 1 liter bag, at pressures of approximately 150 to 900 mmHg.

Since fluid delivery is at the aforementioned pressures, the apparatus can be placed at any level with respect to the patient, as it does not need the assistance of gravity for proper fluid delivery. Moreover, this pressure is sufficient, such that the apparatus is effective and not altitude dependent.

Once at the handpiece, or valving unit 28, the surgeon can control fluid flow into the patient 30 by pressing a button 32, opening or closing a valve (depending on the default position of the valve) in the valving unit 28 (typically, the default position is typically closed, such that fluid is not entering the body and suction is not active on the body until the respective valve is opened). Also, the surgeon can regulate suction by depressing a second button 33, controlling a valve on tubing 34 attached to a suction source (not shown).

The apparatus 22 can be for example, of a size, and dimensioned to accommodate a 1 liter bag of irrigant. However, it can be sized and dimensioned to accommodate various fluid sources, for example, in 3 liter bags (or other similar containers), or any other size desired.

Alternately, the fluid of the fluid source 24 can be blood or blood components, distension fluid or any other fluid usable by the body (for example, in bags or other suitable containers as detailed herein). Connections and tubing for the bag would be in accordance with standard tubing, for example, similar to that detailed herein, for delivering the requisite fluid to the requisite site. For example, a bag of blood as the fluid source 24, typically in 100 ml or 200 ml bags, if placed into the apparatus 22 could be used in emergency situations such as battlefields, emergency rooms, ambulances, etc., to rapidly deliver blood to the body. Similarly, for example, a bag of distension fluid, for example in 3 liter bags, could be delivered from the apparatus 22 to the knee or other joint for arthroscopic surgery, to the uterus in gynecologic procedures, or the like.

While preferred embodiments of systems, apparatus, components and methods, have been described above, the description of the systems, apparatus, components and methods above is exemplary only. Those skilled in the art will recognize, or be able to ascertain using no more than routine experimentation, many equivalents to the specific embodiments of the invention described herein. Such equivalents are intended to be encompassed by the following claims.

What is claimed is:

1. A fluid delivery apparatus comprising:
an activatable driver;
a first section for at least partially housing the activatable driver;
a second section in communication with the first section, the second section configured for holding at least one fluid container;
a partition intermediate the first section and the second section, the partition movable in response to forces from the driver after the driver has been activated.

2. The apparatus of claim 1, wherein the partition is elastic.

3. The apparatus of claim 1, wherein the partition defines a portion of the first section and a portion of the second section.

4. The apparatus of claim 1, wherein the driver includes at least one cylinder having at least one pressurized gas therein.

5. The apparatus of claim 1, wherein the at least one cylinder includes a seal.

6. The apparatus of claim 5, wherein the seal includes a membrane.

7. The apparatus of claim 6, additionally comprising:
a rod, the rod including a puncturing member, the puncturing member in alignment with the membrane and movable to at least one position for puncturing the membrane.

8. The apparatus of claim 5, wherein the seal includes a member configured for rupturing by being broken.

9. The apparatus of claim 4, wherein said at least one pressurized gas includes carbon dioxide.

10. The apparatus of claim 1, wherein the first section includes an air tight chamber.

11. The apparatus of claim 1, wherein the second section defines a pocket.

12. The apparatus of claim 1, additionally comprising:
a fluid container in the second section.

13. The apparatus of claim 12, wherein the fluid is selected from the group comprising: irrigation fluid, drug solutions, blood, blood component solutions and distension fluid.

14. A system for fluid delivery comprising:
a fluid delivery apparatus comprising:
an activatable driver;
a first section for at least partially housing the activatable driver;
a second section in communication with the first section, the second section configured for holding at least one fluid container;
a partition intermediate the first section and the second section, the partition movable in response to forces from the driver after the driver has been activated;
a valving unit for controlling fluid flow to a patient; and
a first conduit in communication with the fluid delivery apparatus and the valving unit.

15. The system of claim 14, additionally comprising:
a suction source; and
a second conduit in communication with the valving unit and the suction source.

16. The system of claim 15, additionally comprising:
a handpiece configured for manual manipulation, the handpiece including the valving unit.

17. The system of claim 14, additionally comprising:
a fluid container in the second section.

18. The system of claim 17, wherein the fluid is selected from the group comprising:
irrigation fluid, drug solutions, blood, blood component solutions and distension fluid.

19. The system of claim 14, wherein the partition is elastic.

20. The system of claim 14, wherein the partition defines a portion of the first section and a portion of the second section.

21. The system of claim 14, wherein the driver includes at least one cylinder having at least one pressurized gas therein.

22. The system of claim 14, wherein the at least one cylinder includes a seal.

23. The system of claim 22, wherein the seal includes a membrane.

24. The system of claim 23, additionally comprising:
a rod, the rod including a puncturing member, the puncturing member in alignment with the membrane and movable to at least one position for puncturing the membrane.

25. The system of claim 22, wherein the seal includes a member configured for rupturing by being broken.

26. The system of claim 14, wherein said at least one pressurized gas includes carbon dioxide.

27. The system of claim 14, wherein the first section includes an air tight chamber.

28. A method for providing fluid to a mammalian body comprising:
providing fluid delivery apparatus comprising:
an activatable driver;
a first section for at least partially housing the activatable driver;
a second section in communication with the first section, the second section including at least one fluid container; and
a partition intermediate the first section and the second section, the partition movable in response to forces from the driver after the driver has been activated;
providing a pathway for fluid in the fluid delivery apparatus to the mammalian body; and
activating the driver.

29. The method of claim 28, additionally comprising:
positioning the fluid delivery apparatus at a level below the mammalian body.

30. The method of claim 28, additionally comprising:
positioning the fluid delivery apparatus at a level above the mammalian body.

31. The method of claim 28, additionally comprising:
positioning the fluid delivery apparatus at a level approximately even with the level of the mammalian body.

32. The method of claim 28, wherein,
the activatable driver includes, a gas cartridge and a membrane sealing the cartridge; and
the step of activating the driver includes puncturing the membrane.

33. The method of claim 28, wherein,
the activatable driver includes, a gas cartridge and a breakable member sealing the cartridge; and
the step of activating the driver includes separating the breakable member from the cartridge to unseal the cartridge.

34. A method for delivering a fluid solution to a patient in need thereof comprising providing the fluid delivery apparatus of any of claims 1-13.
